(19) United States
(12) Patent Application Publication
(10) Pub. No.: US 2005/0175580 A1
(43) Pub. Date: Aug. 11, 2005

(54) Compositions and methods for enhancing cytokine activity and treating hypotension associated with the administration of cytokines

(76) Inventor: Daniela Salvemini, Chesterfield, MO (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 10/433,290

(22) PCT Filed: Jan. 3, 2002
(86) PCT No.: PCT/US02/00036

(30) Foreign Application Priority Data
Jan. 5, 2001 (US) 60260120

(51) Int. Cl. A61K 38/19; A61K 31/555

(52) U.S. Cl. 424/85.1; 514/184

(57) ABSTRACT

This invention relates to compositions which enhance a mammal's immune response to a cytokine by administering therapeutic amounts of catalysts for the dismutation of superoxide which include superoxide dismutase enzyme (SOD) and small molecular weight organic ligand mimics of that enzyme (SOD mimetics or SODms), alone or in combination with a cytokine. These compositions may be used in methods for enhancing a mammal's immune response to a virus such as the hepatitis C virus or the human immunodeficiency virus (HIV) or to a tumor thereby inhibiting the proliferation of the tumor. Relatedly, these compositions may be used in methods of treating mammals infected with HIV or mammals suffering from Hepatitis C or AIDS. This invention also relates to methods of enhancing cancer therapy and methods of preventing and treating hypotension in a mammal resulting from the administration of cytokines by the administration of therapeutic amounts of catalysts for the dismutation of superoxide in combination with a cytokine and catecholamine pressor agents. Also provided are pharmaceutical compositions comprising catalysts for the dismutation of superoxide alone or in combination with cytokines and catecholamine pressor agents for use in the above methods.

### FIELD OF INVENTION

This invention relates to compositions and methods for enhancing the immune activity of a cytokine by co-administering the cytokine with therapeutic amounts of catalysts for the dismutation of superoxide. This invention also relates to compositions and methods of preventing and treating hypotension in a mammal resulting from the administration of cytokines by the administration of therapeutic amounts of catalysts for the dismutation of superoxide in combination with a cytokine. The invention also relates to methods of enhancing cancer treatments in a subject by the administration of therapeutic amounts of catalysts for the dismutation of superoxide in combination with a cytokine. Also provided are pharmaceutical compositions comprising cytokines and catalysts for the dismutation of superoxide alone or in combination with catecholamine pressor agents for use in these methods.

### BACKGROUND OF THE INVENTION

In the immune system, the three major types of lymphocytes are B cells, T cells, and natural killer (NK) cells. B-cells are derived from bone marrow and comprise about 10% of the lymphocytes found circulating in blood. When stimulated by a specific antigen, each B-cell differentiates into a plasma cell that secretes antibodies of a single specificity. T-cells mature in the thymus and make up about 80% of circulating lymphocytes. T-cells have specific antigen receptors resembling antibody molecules on their surfaces and react to antigen stimulation by secreting immunomediator molecules or cytokines and toxic molecules. Cytotoxic T-cells secrete toxic molecules that kill infected cells and any foreign particles that they may contain, such as microorganisms. NK cells make up about 10% of the lymphocyte population, are not antigen specific and recognize and kill cells infected with microbes.

Monocytes and macrophages are large scavenger cells that ingest foreign particles and present antigens to the T-cells which trigger specific immune responses. When an antigen is introduced, it is initially ingested by macrophages and other antigen presenting cells. After digestion, short segments thereof are presented on their cell surfaces. Only a few of all circulating T-cells have receptors that specifically bind to the antigen and this binding stimulates the T-cells to secrete cytokines.

Cytokines are small proteins secreted primarily by cells of the immune system that promote the proliferation and/or differentiative functions of other cells and play a significant rold in the regulation of the magnitude of the immune response. Examples of cytokines include interleukins, interferons, hematopoietic colony stimulating factors (CSF) and proinflammatory factors such as tumor necrosis factor (TNF). Such cytokines act on lymphocytes by stimulating them to proliferate thereby boosting a host's immune response. For example, IL-2 potentiates both innate or natural host defenses by stimulating NK cells and antigen-specific acquired immune reactivity by stimulating T cells and B cells.

Thus, the enhancement of an individual's immunity is a desirable goal in the treatment of patients diagnosed with cancer, autoimmune deficiency syndrome (AIDS), hepatitis and other disease states. Low dosages e.g., about 10-100 IU, of cytokines may be administered to patients in order to produce the desired immune response-enhancing ability of a cytokine in a host.

Additionally, the administration of lymphokines and related immunomodulators is a common therapy in many cancer treatments and has resulted in some positive responses in patients with various types of tumors. For example, high-dosages of interleukin 2 (IL-2) has demonstrated clinical activity in metastatic renal cell carcinoma and malignant melanoma with a small (5-15%) percentage of long term complete responses.

Interleukin-2 (IL-2), a cytokine which is primarily synthesized by activated T lymphocytes, plays a central role in the development of cell-mediated immunity. See Mertelsmann R. and Welte, K., Immunobiol. 172: 400-419 (1988). Low concentrations (e.g., 10-100 IU/ml) of IL-2 appear to be critical for the activation of antigen specific cytolytic lymphocytes as well as the clonal expansion of these effector cells. See Weiss, A., Fundamental Immunology, 3rd ed. New York: Raven Press, 467-504 (1993). Lotze et al. have shown that the exposure of murine or human lymphocytes to high concentrations of IL-2 (>600 IU/ml) over 3-4 days either in vitro or in vivo results in the proliferation of a population of cytotoxic lymphocytes termed lymphokine-activated killer (LAK) cells. See Lotze et al., Cancer Res., 41:4420-4425 (1981). Once activated, LAK cells demonstrate cytotoxicity against a wide variety of freshly isolated cancer cells or cultured cancer cell lines derived from syngeneic, allogeneic or even xenogeneic hosts. See Rayner et al., J. Natl. Cancer Inst., 75: 67-75(1985); Hank et al., Cancer Res. 48: 1965-1971 (1988). Since the pattern of cytotoxicity is not tumor specific and does not require the expression of self-major histocompatibility complex (MHC) on target cells for cytotoxicity, LAK cells are termed “non-specific” killer cells. A major subset of the cytolytic cells in the LAK population are CD3⁻ and express CD56, a neural cell adhesion molecule (N-CAM) thus indicating natural killer (NK) lymphocyte derivation. See Lanier et al., J. Immunol. 136: 4480-4486 (1986). Almost all freshly isolated and cultured malignant cells including multidrug-resistant tumor cells, are susceptible to LAK mediated cytolysis. See Harker et al., Cancer Res., 50: 5931-5936 (1990); Brune and Lapetina, Adv. Pharmacol. 34: 351-360 (1995).

Further, IL-2 is an important lymphokine in the generation of anti-tumor immunity. In response to tumor antigens, a subset of lymphocytes, helper T cells, secrete a small amount of IL-2 which acts locally at the site of tumor antigen stimulation to activate cytotoxic T cells and natural killer cells that mediate systemic tumor cell destruction. It has been observed that the administration of IL-2 is able to induce the regression of certain cancers present in a mouse and in patients having metastatic cancers such as melanoma, cancer of the kidney, colorectal cancer or non-Hodgkin's lymphoma. See Rosenberg et al., N. Engl. J. Med. 316: 889-897 (1987). Further, clinical trials using IL-2 in combination with cytoreductive chemotherapy show promise in treatment of acute myelogenous leukemia, non-hodgkin's lymphoma and breast cancer.

Thus, the modulation of cytokine concentrations has been attempted as a means to enhance a patient's immune response. the administration of low dosages of cytokines such as IL-2 have been shown to stimulate or maintain a patient's immune response. See U.S. Pat. No. 6,045,788. However, the clinical use of high dosages e.g., 100,000 to 600,000 IU, of cytokines such as IL-2, TNF-α, and interleukin-12 is limited by development of severe toxic side effects such as dose-dependent hypotension (systolic blood pressure (SBP)<90 mm/Hg). The toxicity of systemically administered cytokines is not surprising as these agents mediate local cellular interactions and normally are secreted only in very small quantities. Unfortunately, the potentially lethal hypotensive side effect of therapeutic cytokine compositions severely limits the maximum dose of these compounds which can be given to a patient, thus reducing the immune activity and therapeutic anti-tumor effects of the cytokine.

Reactive oxygen species (ROS) and in particular, superoxide anions (O₂⁻) contribute to the unwanted side effects associated with cytokine therapy, such as hypotension, hyporeactivity and reduced T lymphocyte activation. Hypotension is a hemodynamic condition resulting from reduced vascular resistance despite increased levels of endogenous catecholamines. This condition persists despite the maintenance of normal blood volume (normovolemia). A characteristic of this condition is hyporeactivity, the loss of vascular responses, which develops to both exogenous and presumably, endogenous catecholamines.

Another characteristic of hypotension is the large increase in the production of free radicals, including superoxide anions within the body. See Ischiropoulos et al., Arch. Biochem. Biophys. 298: 446-451 (1992); Taylor et al., Arch. Biochem. Biophys., 316: 70-76 (1995). Administration of cytokines such as IL-2 releases superoxide anions which interact with and destroy the biological activity of vasopressor catecholamines, namely norepinephrine, epinephrine and dopamine. Superoxide anions are normally removed in biological systems by the formation of hydrogen peroxide and oxygen in the following reaction (hereinafter referred to as dismutation):
O₂⁻+O₂⁻+2H⁺→O₂+H₂O₂

This reaction is catalyzed in vivo by the ubiquitous superoxide dismutase enzymes. Several non-proteinaceous catalysts which mimic this superoxide dismutating activity have been discovered. A particularly effective family of non-proteinaceous catalysts for the dismutation of superoxide consists of the manganese(II), manganese(III), iron(II) or iron(III) complexes of nitrogen-containing fifteen-membered macrocyclic ligands which catalyze the conversion of superoxide into oxygen and hydrogen peroxide, as described in U.S. Pat. Nos. 5,874,421 and 5,637,578, all of which are incorporated herein by reference. See also, Weiss, R. H., et al., “Manganese(II)-Based Superoxide Dismutase Mimetics: Rational Drug Design of Artificial Enzymes”, Drugs of the Future 21: 383-389 (1996); and Riley, D. P., et al., “Rational Design of Synthetic Enzymes and Their Potential Utility as Human Pharmaceuticals” (1997) in CatTech, I, 41. These mimics of superoxide dismutase have been shown to have a variety of therapeutic effects, including anti-inflammatory activity. See Weiss, R. H., et al., “Therapeutic Aspects of Manganese (II)-Based Superoxide Dismutase Mimics” in “Inorganic Chemistry in Medicine”, (Farrell, N., Ed.), Royal Society of Chemistry, in Press; Weiss, R. H., et al., “Manganese-Based Superoxide Dismutase Mimics: Design, Discovery and Pharmacologic Efficacies” (1995), in “The Oxygen Paradox” (Davies, K. J. A., and Ursini, F., Eds.) pp. 641-651, CLEUP University Press, Padova, Italy; Weiss, R. H., et al., J. Biol. Chem., 271: 26149 (1996); and Hardy, M. M., et al., J. Biol. Chem. 269: 18535-18540 (1994). Other non-proteinaceous catalysts which have been shown to have superoxide dismutating activity are the salen-transition metal cation complexes, as described in U.S. Pat. No. 5,696,109 and complexes of porphyrins with iron and manganese cations.

Currently, management of hypotension associated with cytokine administration includes infusion of fluids and albumin, and in some cases, treatment with catecholamine pressor agents such as dopamine. When dopamine fails to reverse hypotension, epinephrine, phenylephrine or noreprinephrine may be administered. However, despite repeated catecholamine doses, maintenance of an acceptable blood pressure (usually >90 mmHg) is often unattainable. Accordingly, in 20-50% of cases, intensive care unit (ICU) hospitalization for hemodynamic support is required as a result of unresponsive hypotension and hyporeactivity (loss of response to vasoconstrictors). As a result of the dose-limiting side effects, the full period of IL-2 administration is frequently curtailed in order to diminish hypotension and subsequent renal dysfunction.

Accordingly, a need presently exists for compositions and methods for enhancing the immune response resulting from the administration of a cytokine. Also needed are compositions and methods for preventing or treating hypotension which do not adversely affect the therapeutic mechanism of a cytokine. Further needed are compositions and methods for preventing and treating mammals suffering from hypotension resulting from cytokine administration by preventing the decrease of mean arterial pressure or by preventing and reversing the continued decrease of mean arterial pressure. Such compositions and methods would markedly facilitate IL-2 administration and would serve to broaden the clinical use of cytokines. Further needed are compositions and methods for enhancing the effectiveness of cytokine administration for cancer therapy by boosting the immune enhancing activity of the cytokine and/or by preventing and treating hypotension in mammals resulting from the administration of a cytokine.

### SUMMARY OF THE INVENTION

Accordingly, an object of the present invention is to provide pharmaceutical and veterinary compounds and methods which enhance the cytokine-mediated immune response in a host thereby resulting in an improved immune response to a virus. Another object of the present invention is to reduce the dose-limiting toxicity of hypotension associated with cytokine administration thereby allowing higher doses of a therapeutic cytokine to be administered. As a result, the therapeutic effects of the cytokine is potentiated. Applicants have discovered that treatment with catalysts for the dismutation of superoxide, including superoxide dismutase enzyme (SOD) and small molecular weight organic ligand mimics of that enzyme (SOD mimetics or SODms) results in preventing in vitro deactivation of catecholamines. Moreover, this deactivation appears to account for the hyporeactivity to exogenous catecholamines observed in cases of hypotension, thus suggesting that the deactivation of endogenous norepinephrine by superoxide may contribute significantly to this aspect of the vascular crisis.

Without being limited to any particular theory, one object of the invention is to provide compounds and methods to treat hypotension associated with the administration of a cytokine by removing superoxide, thus protecting exogeneous and endogeneous catecholamines from autooxidation. By preventing or limiting the deactivation of catecholamines, hyporeactivity and hypotension are reversed and the effectiveness of cytokine administration as a therapy for cancer can be improved.

Another object of the present invention is to provide methods of inhibiting a fall in mean arterial blood pressure resulting from the administration of a cytokine in a mammal, preferably a human, by co-administering to the mammal a mean arterial pressure sustaining amount of a catalyst for the dismutation of superoxide in conjunction with a cytokine alone or in combination with a catecholamine pressor agent.

Another object of the present invention is to provide a method for increasing mean arterial pressure in a mammal suffering from hypotension resulting from the administration of a cytokine which comprises co-administering to the mammal a mean arterial pressure increasing amount of a catalyst for the dismutation of superoxide in conjunction with a cytokine alone or in combination with a catecholamine pressor agent.

Another aspect of the invention is directed to the administration of a cancer therapy enhancing amount of a catalyst for the dismutation of superoxide alone or in combination with a cytokine and/or a catecholamine pressor agent to a patient undergoing cytokine therapy. Administration of such compounds enhance the tumor-reducing effects of cytokines, preferably IL-2 mediated therapeutic effects, as a cancer therapy. Relatedly, an object of the present invention is to provide methods and compositions for inhibiting the proliferation of a tumor comprising administering a therapeutically effective amount of a catalyst for the dismutation of superoxide alone or in combination with a cytokine.

Another aspect of the invention is directed to compositions and methods for enhancing a cytokine-mediated immune response against a virus comprising administering a composition comprising a catalyst for the dismutation of superoxide alone or in combination with a cytokine. Preferably, the methods and compositions are directed to enhancing the immune response against a hepatitis C virus or a human immunodeficiency virus (HIV).

Relatedly, pharmaceutical compositions are provided which comprise a catalyst for the dismutation of superoxide alone or in combination with a cytokine and a pharmaceutically acceptable carrier. Also provided are pharmaceutical compositions which comprise a catalyst for the dismutation of superoxide, a cytokine, a catecholamine pressor agent and a pharmaceutically acceptable carrier. When administered to a mammal afflicted with a tumor, these compositions enhance the immune response of the patient and the anti-tumor effect of cytokine therapy. Further, these pharmaceutical compositions inhibit the degradation of the catecholamines, allowing the catecholamine pressor agent to improve vascular tone and to increase the mean arterial blood pressure of the mammal. Preferably, the cytokines used in the above methods and compositions are selected from interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), gamma-interferon (γ-IF), interferon-alpha (IF-α), tumor necrosis factor (TNF), granulocyte colony stimulating factor and granulocyte-macrophage colony stimulating factor, and more preferably, IL-2.

Other objects and features will be in part apparent and in part pointed out hereinafter.

### BRIEF DESCRIPTION OF THE DRAWINGS

FIG. 1 is a graph indicating that IL-2 induced hypotension was attenuated in a dose-dependent manner by SOD mimetic M40403 with maximal reversal obtained at 3 mg/kg M40403. At the highest dose of M40403 (3 mg/kg, twice a day (b.i.d.) for 5 days) there were no apparent signs of toxicity and particularly no effect on systolic blood pressure.

FIG. 2 is a graph indicating that higher doses of IL-2 could be administered (M40403 allowed BP to be maintained while IL-2 mediated death rates were reduced) in the presence of M40403. In the absence of M40403, there was 1 death in each IL-2 treated group at 200,000; 250,000 and 300,000 IU bid (1 out 6 died), all mice died in the 400,000 IU bid group (6 deaths of 6 mice). In the presence of M40403, all mice at all doses of IL-2 were alive (0 deaths of 6 mice in all groups).

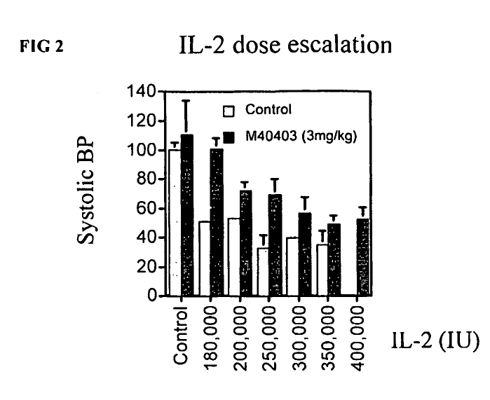

FIG. 3 is a graph indicating that a broad range of M40403 concentrations (0-10 μM) do not adversely affect murine (mouse) LAK cell activation.

FIG. 4 is a graph which shows that SOD mimetic M40403 significantly potentiated IL-2 induced LAK cells activation in vivo as seen in the lymph nodes (A) and spleen (13). M40403, by itself did not increase LAK cell induction in vivo.

FIG. 5 is a graph which indicates that IL-2 treatment of mice eliminated the number of tumor nodules in lungs and does not affect the anti-tumor effects of IL-2. M40403 by itself at the dose used completely eliminated the numbers of tumor nodules in lungs of mice.

FIG. 6 is a graph indicating a survival advantage in IL-2 treated mice, compared to controls (14 days compared to 19 days). M40403 itself did not affect survival. These results suggest that M40403 not only blocks IL-2 induced hypotension but also improves IL-2 induced anti-tumor responses in a highly refractory treatment model.

FIG. 7 is a graph which shows that M40401 did not interfere with the ability of IL-2 to activate LAK cells in vitro and that M40401 reversed IL-2 mediated hypotension at lower doses than M40403. Full reversal of hypotension was seen at 0.3 mg/kg (b.i.d with IL-2 for 5 days, protocol identical to the one used for M40403).

### DEFINITIONS AND ABBREVIATIONS

To facilitate understanding of the invention, a number of terms as used herein are defined below:

The term “hypotension” means a hemodynamic condition characterized by lowered blood pressure which persists despite the maintenance of normal blood volume (normovolemia). Generally, hypotension occurs if the mean arterial pressure is less than 90 mmHg for at least one hour despite adequate ventricular filling pressures (pulmonary artery wedge pressure [PAWP] of 12 mmHg), or despite a sufficient central venous pressure (CVP) of 12 mmHg. Other indicators of hypotension are the failure of the hypotensive state to respond to aggressive initial fluid therapy (such as the administration of 500 ml of isotonic crystalloid, 25 gm or albumin, or 200 ml of other colloids (e.g. hydroxyethyl starch)), or the need for pressor doses of dopamine (>5 g/kg/min), norepinephrine or other pressor agents to maintain a systolic blood pressure of 90 mmHg.

As used herein, “MAP” refers to mean arterial pressure.

The term “mean arterial pressure sustaining amount”, as used in this application, means the amount of a compound needed to maintain the mean arterial pressure of a mammal suffering from or at imminent risk of suffering from hypotension associated with various shock states in the normotensive range, preferably from about 70 to about 130 mmHg for at least 30 minutes.

The term “mean arterial pressure increasing amount”, as used in this application, means the amount of a compound needed to increase the mean arterial pressure of a mammal suffering from hypotension associated with various shock states from its hypotensive state to the normotensive range, preferably from about 70 to about 130 mmHg for at least 30 minutes.

The phrase “co-administering a catalyst for the dismutation of superoxide in conjunction with a cytokine” means that the catalyst is administered contemporaneous with, or sufficiently close in time before or after administration of the cytokine so as to inhibit the hypotensive effects caused by cytokine administration and/or, as the context requires, sufficiently close in time to enhance cytokine-mediated therapeutic effects on the mammal's immune system and to inhibit the proliferation of a tumor.

The term “non-proteinaceous catalysts for the dismutation of superoxide” means a low-molecular weight catalyst for the conversion of superoxide anions into hydrogen peroxide and molecular oxygen. These catalysts commonly consist of an organic ligand and a chelated transition metal ion, preferably manganese(II), manganese(III), iron(II) or iron(III). The term may include catalysts containing short-chain polypeptides (under 15 amino acids), or macrocyclic structures derived from amino acids, as the organic ligand. The term explicitly excludes a superoxide dismutase enzyme obtained from any species.

The term “substituted” means that the described moiety has one or more substituents comprising at least 1 carbon or heteroatom, and further comprising 0 to 22 carbon atoms, more preferably from 1 to 15 carbon atoms, and comprising 0 to 22, more preferably from 0 to 15. As used herein, “heteroatom” refers to those atoms that are neither carbon nor hydrogen bound to carbon and are selected from the group consisting of: O, S, N, P, Si, B, F, Cl, Br, or I. These atoms may be arranged in a number of configurations, creating substituent groups which are unsaturated, saturated, or aromatic. Examples of such substituents include branched or unbranched alkyl, alkenyl, or alkynyl, cyclic, heterocyclic, aryl, heteroaryl, allyl, polycycloalkyl, polycycloaryl, polycycloheteroaryl, imines, aminoalkyl, hydroxyalkyl, hydroxyl, phenol, amine oxides, thioalkyl, carboalkoxyalkyl, carboxylic acids and their derivatives, keto, ether, aldehyde, amine, amide, nitrile, halo, thiol, sulfoxide, sulfone, sulfonic acid, sulfide, disulfide, phosphonic acid, phosphinic acid, acrylic acid, sulphonamides, amino acids, peptides, proteins, carbohydrates, nucleic acids, fatty acids, lipids, nitro, hydroxylamines, hydroxamic acids, thiocarbonyls, thiocarbonyls, borates, boranes, boraza, silyl, silaza, siloxy, and combinations thereof.

The term “alkyl”, alone or in combination, means a straight-chain or branched-chain alkyl radical containing from 1 to about 22 carbon atoms, preferably from about 1 to about 18 carbon atoms, and most preferably from about 1 to about 12 carbon atoms. Examples of such radicals include, but are not limited to, methyl, ethyl, n-propyl, isopropyl, n-butyl, isobutyl, sec-butyl, tert-butyl, pentyl, iso-amyl, hexyl, octyl, nonyl, decyl, dodecyl, tetradecyl, hexadecyl, octadecyl and eicosyl.

The term “alkenyl”, alone or in combination, means an alkyl radical having one or more double bonds. Examples of such alkenyl radicals include, but are not limited to, ethenyl, propenyl, 1-butenyl, cis-2-butenyl, trans-2-butenyl, iso-butylenyl, cis-2-pentenyl, trans-2-pentenyl, 3-methyl-1-butenyl, 2,3-dimethyl-2-butenyl, 1-pentenyl, 1-hexenyl, 1-octenyl, decenyl, dodecenyl, tetradecenyl, hexadecenyl, cis- and trans-9-octadecenyl, 1,3-pentadienyl, 2,4-pentadienyl, 2,3-pentadienyl, 1,3-hexadienyl, 2,4-hexadienyl, 5,8,11,14-eicosatetraenyl, and 9,12,15-octadecatrienyl.

The term “alkynyl”, alone or in combination, means an alkyl radical having one or more triple bonds. Examples of such alkynyl groups include, but are not limited to, ethynyl, propynyl (propargyl), 1-butynyl, 1-octynyl, 9-octadecynyl, 1,3-pentadiynyl, 2,4-pentadiynyl, 1,3-hexadiynyl, and 2,4-hexadiynyl.

The term “cycloalkyl”, alone or in combination means a cycloalkyl radical containing from 3 to about 10, preferably from 3 to about 8, and most preferably from 3 to about 6, carbon atoms. Examples of such cycloalkyl radicals include, but are not limited to, cyclopropyl, cyclobutyl, cyclopentyl, cyclohexyl, cycloheptyl, cyclooctyl, and perhydronaphthyl.

The term “cycloalkylalkyl” means an alkyl radical as defined above which is substituted by a cycloalkyl radical as defined above. Examples of cycloalkylalkyl radicals include, but are not limited to, cyclohexylmethyl, cyclopentylmethyl, (4-isopropylcyclohexyl)methyl, (4-t-butyl-cyclohexyl)methyl, 3-cyclohexylpropyl, 2-cyclohexylmethylpentyl, 3-cyclopentylmethylhexyl, 1-(4-neopentylcyclohexyl)methylhexyl, and 1-(4-isopropylcyclohexyl)methylheptyl.

The term “cycloalkylcycloalkyl” means a cycloalkyl radical as defined above which is substituted by another cycloalkyl radical as defined above. Examples of cycloalkylcycloalkyl radicals include, but are not limited to, cyclohexylcyclopentyl and cyclohexylcyclohexyl.

The term “cycloalkenyl”, alone or in combination, means a cycloalkyl radical having one or more double bonds. Examples of cycloalkenyl radicals include, but are not limited to, cyclopentenyl, cyclohexenyl, cyclooctenyl, cyclopentadienyl, cyclohexadienyl and cyclooctadienyl.

The term “cycloalkenylalkyl” means an alkyl radical as defined above which is substituted by a cycloalkenyl radical as defined above. Examples of cycloalkenylalkyl radicals include, but are not limited to, 2-cyclohexen-1-ylmethyl, 1-cyclopenten-1-ylmethyl, 2-(1-cyclohexen-1-yl)ethyl, 3-(1-cyclopenten-1-yl)propyl, 1-(1-cyclohexen-1-ylmethyl)pentyl, 1-(1-cyclopenten-1-yl)hexyl, 6-(1-cyclohexen-1-yl)hexyl, 1-(1-cyclopenten-1-yl)nonyl and 1-(1-cyclohexen-1-yl)nonyl.

The terms “alkylcycloalkyl” and “alkenylcycloalkyl” mean a cycloalkyl radical as defined above which is substituted by an alkyl or alkenyl radical as defined above. Examples of alkylcycloalkyl and alkenylcycloalkyl radicals include, but are not limited to, 2-ethylcyclobutyl, 1-methylcyclopentyl, 1-hexylcyclopentyl, 1-methylcyclohexyl, 1-(9-octadecenyl)cyclopentyl and 1-(9-octadecenyl)cyclohexyl.

The terms “alkylcycloalkenyl” and “alkenylcycloalkenyl” means a cycloalkenyl radical as defined above which is substituted by an alkyl or alkenyl radical as defined above. Examples of alkylcycloalkenyl and alkenylcycloalkenyl radicals include, but are not limited to, 1-methyl-2-cyclopentyl, 1-hexyl-2-cyclopentenyl, 1-ethyl-2-cyclohexenyl, 1-butyl-2-cyclohexenyl, 1-(9-octadecenyl)-2-cyclohexenyl and 1-(2-pentenyl)-2-cyclohexenyl.

The term “aryl”, alone or in combination, means a phenyl or naphthyl radical which optionally carries one or more substituents selected from alkyl, cycloalkyl, cycloalkenyl, aryl, heterocycle, alkoxyaryl, alkaryl, alkoxy, halogen, hydroxy, amine, cyano, nitro, alkylthio, phenoxy, ether, trifluoromethyl and the like, such as phenyl, p-tolyl, 4-methoxyphenyl, 4-(tert-butoxy)phenyl, 4-fluorophenyl, 4-chlorophenyl, 4-hydroxyphenyl, 1-naphthyl, 2-naphthyl, and the like.

The term “aralkyl”, alone or in combination, means an alkyl or cycloalkyl radical as defined above in which one hydrogen atom is replaced by an aryl radical as defined above, such as benzyl, 2-phenylethyl, and the like.

The term “heterocyclic” means ring structures containing at least one heteroatom within the ring. As used herein, “heteroatom” refer to atoms that are neither carbon nor hydrogen bound to a carbon. Examples of heterocyclics include, but are not limited to, pyrrolidinyl, piperidyl, imidazolidinyl, tetrahydrofuryl, tetrahydrothienyl, furyl, thienyl, pyridyl, quinolyl, isoquinolyl, pyridazinyl, pyrazinyl, indolyl, imidazolyl, oxazolyl, thiazolyl, pyrazolyl, pyridinyl, benzoxadiazolyl, benzothiadiazolyl, triazolyl and tetrazolyl groups.

The term “saturated, partially saturated or unsaturated cyclic” means fused ring structures in which 2 carbons of the ring are also part of the fifteen-membered macrocyclic ligand. The ring structure can contain 3 to 20 carbon atoms, preferably 5 to 10 carbon atoms, and can also contain one or more other kinds of atoms in addition to carbon. The most common of the other kinds of atoms include nitrogen, oxygen and sulfur. The ring structure can also contain more than one ring.

The term “saturated, partially saturated or unsaturated ring structure” means a ring structure in which one carbon of the ring is also part of the fifteen-membered macrocyclic ligand. The ring structure can contain 3 to 20, preferably 5 to 10, carbon atoms and can also contain nitrogen, oxygen and/or sulfur atoms.

The term “nitrogen containing heterocycle” means ring structures in which 2 carbons and a nitrogen of the ring are also part of the fifteen-membered macrocyclic ligand. The ring structure can contain 2 to 20, preferably 4 to 10, carbon atoms, can be substituted or unsubstituted, partially or fully unsaturated or saturated, and can also contain nitrogen, oxygen and/or sulfur atoms in the portion of the ring which is not also part of the fifteen-membered macrocyclic ligand.

The term “organic acid anion” refers to carboxylic acid anions having from about 1 to about 18 carbon atoms.

The term “halide” means chloride, fluoride, iodide, or bromide.

As used herein, “R” groups means all of the R groups attached to the carbon atoms of the macrocycle, i.e., R, R′, R₁, R′₁, R₂, R′₂, R₃, R′₃, R₄, R′₄, R₅, R′₅, R₆, R′₆, R₇, R′₇, R₈, R′₈, R₉, R′₉.

The term “cancer therapy” refers to any treatment designed to kill tumor cells in a patient. As used herein, the terms “tumor cell” and “cancer cell” are used interchangeably to mean a malignant cell. A tumor cell can occur in and can be obtained from a solid tumor such as a sarcoma, carcinoma, melanoma, lymphoma or glioma or a more diffuse cancer such as a leukemia.

The mammal patient in the methods of the invention is a mammal suffering from hypotension resulting from the administration of a cytokine or at imminent risk of hypotension due to the future administration of a cytokine. The term “mammal suffering from hypotension” is thus contemplated to include cases in which hypotension is anticipated, such as with imminent cytokine administration, as well as cases in which hypotension is apparent e.g., after cytokine administration. It is envisioned that a mammal patient to which the catalyst for the dismutation of superoxide will be administered, in the methods or compositions of the invention, will be a human. However, other mammal patients in veterinary (e.g., companion pets and large veterinary animals) and other conceivable contexts are also contemplated.

As used herein, the term “cytokine” refers to a member of the class of proteins that are produced by cells of the immune system and positively regulate or modulate effector functions of the immune response. Such regulation can occur within the humoral or the cell mediated immune response and can modulate the effector functions of T cells, B cells, macrophages, antigen presenting cells or other immune system cells. Specific examples of cytokines include, for example, interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), gamma-interferon (γ-IF), interferon-alpha (IF-α) tumor necrosis factor (TNF), granulocyte colony stimulating factor and granulocyte-macrophage colony stimulating factor.

As used herein, the terms “treatment” or “treating” relate to any treatment of hypotension and include: (I) preventing hypotension from occurring in a subject; (2) inhibiting the fall of mean arterial pressure, i.e., arresting or limiting its development; or (3) ameliorating or relieving the symptoms of the disease. Additionally, as the context requires, the terms “treatment” or “treating” relate to any treatment of a subject for viral infection and include: (1) preventing viral infection from occurring in a subject; (2) inhibiting, arresting or limiting the development of the virus in the subject; or (3) ameliorating or reliving the symptoms of the viral infection.

All references cited herein are explicitly incorporated by reference.

### DETAILED DESCRIPTION

This invention relates to compositions and methods for enhancing the immune-mediated activity of a cytokine by administering therapeutic amounts of catalysts for the dismutation of superoxide alone or in combination with a low dosage of a cytokine. Relatedly, the present invention is directed to methods and compositions for the prevention and treatment of hypotension associated with the administration of high dosages of a cytokine which comprises co-administering compositions containing a catalyst for the dismutation of superoxide in conjunction with a cytokine. Preferred catalysts include superoxide dismutase enzyme (SOD) and small molecular weight organic ligand mimics of that enzyme (SOD mimetics or SODms).

Recent studies have indicated that macrophage synthesis of reactive oxygen species (ROS) such as superoxide ion (° 2′) may be a factor which inhibits the LAK cell activation process. Reactive oxygen species may be induced, in part as a result of inflammatory cytokines produced by LAK cells. The production of such inflammatory cytokines results in the upregulation of xanthine oxidase, an enzyme which generates superoxide as a byproduct. Thus, administration of cytokines such as IL-2 to a patient in need thereof releases superoxide ions which have been shown to interact and destroy the biological activity of vasopressor catecholamines e.g., norepinephrine, epinephrine and dopamine. Moreover, Applicants believe that superoxide (O₂⁻) reacts with catecholamines initiating a chain autooxidation reaction and deactivating them in vitro. Moreover, this deactivation may account for the hyporeactivity to exogenous catecholamines observed in cases of hypotension associated with cytokine administration. This suggests that the deactivation of endogenous norepinephrine by O₂⁻ contributes significantly to this aspect of the vascular crisis. Applicants have found that administration of a catalyst for the dismutation of superoxide protects these catecholamines from degradation and prevents both the hypotension and hyporeactivity associated with various shock states. See Macarthur et al., PNAS 97:9753 (2000).

Further, reactive oxygen species induce NF_{κ}B activation which may depress autocrine production of IL-2 and other cytokines that amplify LAK cell expansion. See Ginn et al., Free Radic. Biol. Med. 25: 346-361 (1998). Exposure of lymphocytes to oxidative radicals such as superoxide may result in lymphocyte anergy and apoptosis which in turn, limits the anti-tumor efficacy of IL-2 and other cytokines. Accordingly, the removal of O₂⁻ will prevent and reduce the dose-limiting toxicity of hypotension resulting from cytokine administration, thus allowing for continued administration of such cytokines. Further, the removal of superoxide will increase cytokine mediated LAK cell induction and potentiate the effectiveness of IL-2 and other cytokines as a cancer agent. Applicants believe that increased LAK cell activity may result in increased cytotoxicity by natural killer (NK) cells and perhaps T lymphocytes. An 8-18 fold increase in tumor cell killing (either via expansion of cytolytic cell numbers or via increased efficiency) could be translated into increased numbers of tumor cells killed and greater anti-cancer responses in vivo. Additionally, increased LAK cell activity may result in a possible shift towards increased TH1 helper cell response (pro-cytotoxic). The TH1 helper cell is a subset of helper T cells which may dictate the emergence of predominantly cytolytic immune responses versus predominantly antibody/DTH mediated responses based on cytokine secretion patterns. Induction of cytotoxicity is facilitated by IL-2, IFNγ, IL-12 (TH1 cytokine), and antibody responses appear to be induced by IL-4, IL-5, IL-6 with IL-10 acting to decrease TH1 activity (TH2 cytokine).

Accordingly, in one embodiment of the invention, the present methods and compositions include cytokines and catalysts for the dismutation of superoxide to treat hypotension resulting from administration of a cytokine. As a result, O₂⁻ is removed, hyporeactivity and hypotension associated with cytokine administration are reversed, and survival rate is improved. Depending on the condition of the mammal and the therapy used, the catalyst is administered before the administration of the cytokine, administered contemporaneously with the cytokine or administered shortly after the administration of the cytokine. Without being limited to a particular theory, it is believed that the administration of a composition containing a cytokine and a catalyst for the dismutation of superoxide enhances the cytokine immune response of the mammal to the tumor while preventing the potentially lethal hypotensive side effects associated with cytokine administration.

Relatedly, applicants believe that the administration of a composition containing a catalyst for the dismutation of superoxide in combination with a low dosage of cytokine enhances the ability of the cytokine to stimulate an immune response thereby enhancing the cytokine immune response of the mammal to the tumor. In doing so, the ability of the host to inhibit the proliferation of a tumor is improved. As a result, cytokine administration can be continued and the cancer treatment is enhanced. Preferably, the cytokine used in the above compositions and methods is interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), gamma-interferon (γ-IF), interferon-alpha (IF-α), tumor necrosis factor (TNF), granulocyte colony stimulating factor and granulocyte-macrophage colony stimulating factor, and more preferably, IL-2.

In another embodiment, the cytokine-mediated immune response to a virus, preferably, a hepatitis C or human immunodeficiency virus (HIV) may be enhanced by administering a composition comprising a catalyst for the dismutation of superoxide in combination with a cytokine. Relatedly, Applicants believe that a composition containing a cytokine and a catalyst for the dismutation of superoxide may be administered for the treatment of Hepatitis C. Preferably, the cytokine administered in combination with the catalyst for the dismutation of superoxide is IF-α for the treatment of Hepatitis C. In another embodiment, Applicants believe that a composition containing a cytokine and a catalyst for the dismutation of superoxide may be administered for the treatment of a mammal infected with HIV or suffering from AIDS. In one preferred embodiment, the cytokine administered in combination with the catalyst for the dismutation of superoxide is IL-2 for the treatment of a mammal infected with HIV or suffering from AIDS.

Catalysts for the Dismutation of Superoxide

It is preferred that non-proteinaceous catalysts for the dismutation of superoxide be used in the methods and compositions of the invention. The pentaaza-macrocyclic non-proteinaceous catalysts preferred for use in the invention have catalytic activities which are close to or equal that of the enzymatic catalysts. Unlike the enzymes, the non-proteinaceous catalysts do not degrade in solution when stored for long periods of time at ambient temperatures and are considerably less antigenic. In addition, these catalysts are usually much simpler to synthesize and produce than enzymes, which must be isolated from natural sources or produced using recombinant biotechnology.

Non-proteinaceous catalysts for the dismutation of superoxide for use in the present invention preferably comprise an organic ligand and a transition metal cation. Particularly preferred catalysts are pentaaza-macrocyclic ligand compounds, more specifically the manganese(II), manganese (III), iron(II) and iron(I) chelates of pentaazacyclopentadecane compounds. The pentaaza macrocyclic ligand complexes of Mn(II) are particularly advantageous for use in the present invention because, in addition to a low molecular weight, they are highly selective for the dismutation of super oxide anions and possess catalytic rates similar or faster than native SOD counterparts. These pentaazacyclopentadecane compounds can be represented by the following formula: 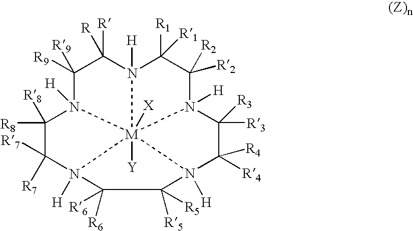
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₂, R₄ or R′₄ and R₅ or R′₅, R₆ or R′₆ and R₇ or R′₇, and R₈ or R′₈, and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉ and R′₉ which is attached to a different carbon atom in the macrocyclic ligand may be bound to form a strap represented by the formula:
—(CH₂)ₓ-M—(CH₂)_{w}-L—(CH₂)_{z}-1-(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof.

Thus, the pentaaza-macrocyclic ligand compounds useful in the present invention can have any combinations of substituted or unsubstituted R groups, saturated, partially saturated or unsaturated cyclics, ring structures, nitrogen containing heterocycles, or straps as defined above.

X, Y and Z represent suitable ligands or charge-neutralizing anions which are derived from any monodentate or polydentate coordinating ligand or ligand system or the corresponding anion thereof (for example benzoic acid or benzoate anion, phenol or phenoxide anion, alcohol or alkoxide anion). X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins. The preferred ligands from which X, Y and Z are selected include halide, organic acid, nitrate and bicarbonate anions.

The “R” groups attached to the carbon atoms of the macrocycle can be in the axial or equatorial position relative to the macrocycle. When the “R” group is other than hydrogen or when two adjacent “R” groups, i.e., on adjacent carbon atoms, together with the carbon atoms to which they are attached form a saturated, partially saturated or unsaturated cyclic or a nitrogen containing heterocycle, or when two R groups on the same carbon atom together with the carbon atom to which they are attached form a saturated, partially saturated or unsaturated ring structure, it is preferred that at least some of the “R” groups are in the equatorial position for reasons of improved activity and stability. This is particularly true when the complex contains more than one “R” group which is not hydrogen.

A wide variety of pentaaza-macrocyclic ligand compounds with superoxide dismutating activity may be readily synthesized. Generally, the transition metal center of the catalyst is thought to be the active site of catalysis, wherein the manganese or iron ion cycles between the (II) and (III) states. Thus, as long as the redox potential of the ion is in a range in which superoxide anion can reduce the oxidized metal and protonated superoxide can oxidize the reduced metal, and steric hindrance of the approach of the superoxide anion is minimal, the catalyst will function with a k_{cat} of about 10⁶ to 10⁸.

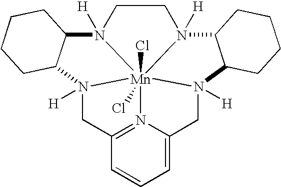

The pentaaza-macrocyclic ligand compound catalysts described have been further described in U.S. Pat. No. 5,637,578, PCT application WO98/58636, and co-pending application U.S. Ser. No. 09/398,120, all of which are hereby incorporated by reference. These pentaaza-macrocyclic ligand catalysts may be produced by the methods disclosed in U.S. Pat. No. 5,610,293. However, it is preferred that the pentaaza-macrocyclic ligand compound catalysts used in the present invention be synthesized by the template method described in copending applications U.S. Ser. No. 60/136,298 and U.S. Ser. No. 09/398,120, hereby incorporated by reference.

A preferred compound of this pentaaza-macrocyclic ligand class is designated compound M40401 and is represented by the following formula:

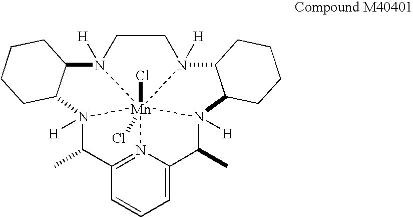

Another preferred compound of this pentaaza-macrocyclic ligand class is designated compound M40403 and is represented by the following formula:

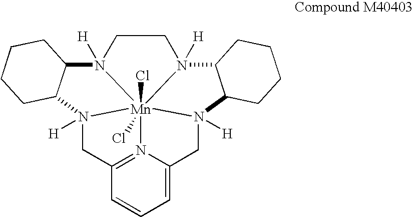

Also suitable for use in the present invention, but less preferred than the pentaaza-macrocyclic ligand compounds, are the salen complexes of manganese and iron disclosed in U.S. Pat. No. 5,696,109, incorporated herein by reference. The term salen complex means a ligand complex with the general formula: 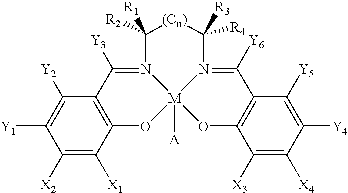
wherein M is a transition metal ion, preferably manganese or iron; A is an anion, typically Cl; and n is either 0, 1, or 2. X₁, X₂, X₃ and X₄ are independently selected from the group consisting of hydrogen, silyls, arlyls, aryls, arylalkyls, primary alkyls, secondary alkyls, tertiary alkyls, alkoxys, aryloxys, aminos, quaternary amines, heteroatoms, and hydrogen; typically X₁ and X₃ are from the same functional group, usually hydrogen, quaternary amine, or tertiary butyl, and X₂ and X₄ are typically hydrogen. Y₁, Y₂, Y₃, Y₄, Y₅, and Y₆ are independently selected from the group consisting of hydrogen, halides, alkyls, aryls, arylalkyls, silyl groups, aminos, alkyls or aryls bearing heteroatoms; aryloxys, alkoxys, and halide; preferably, Y₁ and Y₄ are alkoxy, halide, or amino groups. Typically, Y₁ and Y₄ are the same. R₁, R₂, R₃ and R₄ are independently selected from the group consisting of H, CH₃, C₂H₅, C₆H₅, O-benzyl, primary alkyls, fatty acid esters, substituted alkoxyaryls, heteroatom-bearing aromatic groups, arylalkyls, secondary alkyls, and tertiary alkyls. Methods of synthesizing these salen complexes are also disclosed in U.S. Pat. No. 5,696,109.

Iron or manganese porphyrins, such as, for example, Mn(III) tetrakis(4-N-methylpyridyl)porphyrin, Mn(III) tetrakis-o-(4-N-methylisonicotinamidophenyl)porphyrin, Mn(III) tetrakis(4-N-N-N-trimethylanilinium)porphyrin, Mn(III) tetrakis(1-methyl-4-pyridyl)porphyrin, Mn^{III} tetrakis(4-benzoic acid)porphyrin, Mn(II) octabromo-meso-tetrakis(N-methylpyridinium-4-yl)porphyrin, 5,10,15,20-tetrakis (2,4,6-trimethyl-3,5-disulfonatophenyl)-porphyrinato iron (III) (FeTMPS), Fe(III) tetrakis(4-N-methylpyridyl)porphyrin, and Fe(III) tetrakis-o-(4-N-methylisonicotinamidophenyl)porphyrin and preferably, substituted iron porphyin 5,10,15,20-tetrakis(2,4,6-trimethyl-3,5-disulfonatophenyl)-porphyrinato iron (III) (FeTMPS) may also be used in the methods and compositions of the present invention. See U.S. Pat. No. 6,103,714. The catalytic activities and methods of purifying or synthesizing these non-proteinaceous catalysts are well known in the organic chemistry arts.

In addition to non-proteinaceous catalysts for the dismutation of superoxide, superoxide dismutatse enzymes (SODs) isolated from various sources, or recombinantly produced, may be used in the methods and compositions of the present invention. The best known of these enzymes is CuZn SOD, which is a dimer with a molecular weight of 33,000 containing two copper and two zinc atoms. CuZn SOD is found in the cytosol and in the intermembrane space of the mitochondria. Mn SOD is a tetramer with a molecular weight of 85,000 containing 4 Mn atoms, and is mainly located in the mitochondrial matrix. These enzymes are well known in the biochemical arts, and methods for their isolation and preparation are also well known. See U.S. Pat. No. 5,788,961, incorporated herein by reference. In addition, CuZn SOD is commercially available under the trade name ORGOTEIN (Peroxinorm). As the method of action of the invention is presumed to take place in the walls of the blood vessels of the mammal, the difference in diffusion rates between these enzyme catalysts and the non-proteinaceous catalysts would not be expected to affect the catecholamine preservation effect seen with the intravascular administration of catalysts for the dismutation of superoxide. Thus, these enzyme catalysts would be expected to be effective in the methods and compositions of the invention. However, enzyme catalysts are not preferred, as they can cause allergic reactions in some individuals, are fairly rapidly degraded in the bloodstream, and are much more difficult to produce than their small organic ligand non-proteinaceous counterparts.

Activity of the compounds or complexes of the present invention for catalyzing the dismutation of superoxide can be demonstrated using the stopped-flow kinetic analysis technique as described in Riley, D. P. et al., Anal. Biochem., 196: 344-349 (1991) which is incorporated by reference herein. Stopped-flow kinetic analysis is an accurate and direct method for quantitatively monitoring the decay rates of superoxide in water. The stopped-flow kinetic analysis is suitable for screening compounds for SOD activity and activity of the compounds or complexes of the present invention, as shown by stopped-flow analysis, correlate to treating the above disease states and disorders.

Contemplated equivalents of the general formulas set forth above for the compounds and derivatives as well as the intermediates are compounds otherwise corresponding thereto and having the same general properties such as tautomers of the compounds and such as wherein one or more of the various R groups are simple variations of the substituents as defined therein, e.g., wherein R is a higher alkyl group than that indicated, or where the tosyl groups are other nitrogen or oxygen protecting groups or wherein the O-tosyl is a halide. Anions having a charge other than 1, e.g., carbonate, phosphate, and hydrogen phosphate, can be used instead of anions having a charge of 1, so long as they do not adversely affect the overall activity of the complex. However, using anions having a charge other than 1 will result in a slight modification of the general formula for the complex set forth above. In addition, where a substituent is designated as, or can be, a hydrogen, the exact chemical nature of a substituent which is other than hydrogen at that position, e.g., a hydrocarbyl radical or a halogen, hydroxy, amino and the like functional group, is not critical so long as it does not adversely affect the overall activity and/or synthesis procedure. Further, it is contemplated that manganese(III) complexes will be equivalent to the subject manganese(II) complexes.

In another preferred embodiment, catalysts for the dismutation of superoxide are coupled with catecholamine pressor agents to be used in the compositions and methods for treating hypotension associated with the administration of a cytokine. Preferably, the catecholamine pressor agent is dopamine, norepinephrine, epinephrine and alpha agonist phenyleprine, more preferably, dopamine and norepinephrine. Without being bound to any particular theory, applicants propose that the administration of a composition comprising a cytokine, a catalyst for dismutation of superoxide and a catechol amine pressor agent to a mammal suffering from hypotension resulting from cytokine administration will prevent the degradation of the catecholamines, thus allowing the catecholamine pressor agent to improve vascular tone and increase the mean arterial blood pressure of the mammal. Depending on the condition of the mammal and the therapy used, the catecholamine pressor agent is administered before the administration of the catalyst and cytokine or administrated contemporaneously with the catalyst and cytokine.

Pharmaceutical Compositions

The compounds of the invention can be formulated as pharmaceutical or veterinary compositions for use in the present invention. Depending on the subject to be treated, the mode of administration, and the type of treatment desired (e.g., inhibition, prevention, prophylaxis, therapy), the compounds are formulated in ways consonant with these parameters. The compositions of the present invention comprise a therapeutically or prophylactically effective dosage of a catalyst for the dismutation of superoxide alone or in combination with a cytokine. The catalyst for the dismutation of superoxide is preferably a superoxide dismutase enzyme such as CuZn SOD, or a low molecular weight organic ligand mimics of that enzyme (SODm). In a preferred embodiment, the catalyst is a non-proteineous catalyst comprising an organic ligand and a transition metal cation, more preferably manganese(II), manganese (III), iron (II), and iron(III) chelates of pentaazacyclopentadecane compounds. Also suitable for use in the present invention are the salen complexes of manganese and iron disclosed in U.S. Pat. No. 5,696,109, and iron or manganese porphyrins as discussed above.

In a preferred embodiment, the pharmaceutical or veterinary compositions of the present invention comprise a therapeutically or prophylactically effective dosage of a catalyst for the dismutation of superoxide, administered concurrently with a cytokine. When administered to a mammal, these compositions enhance the ability of a cytokine to stimulate an immune response thereby enhancing the cytokine immune response of the mammal to the tumor. In doing so, the ability of the host to inhibit the proliferation of a tumor is improved. Preferably, the cytokine used in the above compositions and methods is interleukin-1 (IL-1), interleukin-2 (IL-2), interleukin-3 (IL-3), interleukin-4 (IL-4), interleukin-5 (IL-5), interleukin-6 (IL-6), interleukin-7 (IL-7), interleukin-10 (IL-10), interleukin-12 (IL-12), interleukin-15 (IL-15), gamma-interferon (γ-IF), interferon-alpha (IF-α), tumor necrosis factor (TNF), granulocyte colony stimulating factor and granulocyte-macrophage colony stimulating factor, and more preferably, IL-2.

In another embodiment, a composition comprising a catalyst for the dismutation of superoxide and a cytokine may be administered to a mammal to enhance the cytokine-mediated immune response to a virus, preferably, a hepatitis C or human immunodeficiency virus (HIV). Relatedly, such compositions may be administered for the treatment of Hepatitis C or AIDS. The preferred cytokine administered in combination with the catalyst for the dismutation of superoxide is IF-α for the treatment of Hepatitis C and the preferred cytokine administered in combination with the catalyst for the dismutation of superoxide is IL-2 for the treatment of a mammal infected with HIV or suffering from AIDS.

In another preferred embodiment, pharmaceutical or veterinary compositions are provided which comprise catalysts for the dismutation of superoxide, cytokines and catecholamine pressor agents. When administered to a mammal in a state of hypotension resulting from the administration of a cytokine, these pharmaceutical compositions prevent or reduce the potentially lethal hypotension side effect associated with cytokine administration thereby allowing the continued administration of cytokine to treat cancer. Preferably, these compositions prevent the degradation of catecholamines, allowing the catecholamine pressor agent to improve vascular tone and increase the mean arterial blood pressure of the mammal.

The compositions of the present invention may be incorporated in conventional pharmaceutical formulations (e.g. injectable solutions) for use in treating humans or animals in need thereof. Pharmaceutical compositions can be administered by subcutaneous, intravenous, or intramuscular injection, or as large volume parenteral solutions and the like. The term parenteral as used herein includes subcutaneous injections, intravenous, intramuscular, intrasternal injection, or infusion techniques.

For example, a parenteral therapeutic composition may comprise a sterile isotonic saline solution containing between 0.1 percent and 30 percent weight to volume of the catalysts for the dismutation of superoxide. A preferred solution contains from about 05 percent to about 10 percent, more preferably from about 1 percent to about 5 percent, and most preferably about 2.5 percent catalysts for dismutation of superoxide in solution (% weight per volume).

Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in ethanol and water. Among the acceptable vehicles and solvents that may be employed are water and isotonic sodium chloride solution. In addition, sterile, fixed oils are conventionally employed as a solvent or suspending medium.

Suppositories for rectal administration of the drug can be prepared by mixing the drug with a suitable nonirritating excipient such as cocoa butter and polyethylene glycols which are solid at room temperature but liquid at the rectal temperature and will therefore melt in the rectum and release the drug.

For administration to animal or human subjects, a typical dose of the composition comprising a catalyst for the dismutation of superoxide, catalyst for the dismutation of superoxide and a cytokine comprises 0.001 to about 10 mg of a catalyst for the dismutation of superoxide and a catecholamine pressor agent per kilogram of patient body weight and 100 to 600,000 I.U. of cytokine per kilogram of patient body weight. Preferably, the dosage of the catalyst for the dismutation of superoxide and catalyst for the dismutation of superoxide will range between 0.001 to 5 mg/kg patient body weight, more preferably 0.05 to 5 mg/kg body weight, and most preferably 0.05 to 1 mg/kg body weight. Preferably, the dosage of the cytokine in the composition will range between about 500 to about 100,000 I.U./kg body weight, more preferably about 1,000 to about 100,000 I.U./kg body weight and most preferably, about 5,000 to about 100,000 I.U./kg body weight. In another aspect of the invention, compositions may be administered to a patient to enhance the cytokine-mediated response against a virus. The typical dosages of such compositions comprising a catalyst for the dismutation of superoxide alone or in combination with a cytokine comprises 0.001 to about 10 mg of a catalyst for the dismutation of superoxide and preferably, 10-100 I.U. of cytokine per kilogram of patient body weight. For the above compositions, the total daily dose may be administered to a mammal in single or divided doses. Dosage unit compositions may contain such amounts of submultiples thereof to make up the total dose. However, one skilled in the art will recognize that the total dosage will vary on the particular composition being administered.

The amount of active ingredient that may be combined with the carrier materials to produce a single dosage form will vary depending upon the host treated and the particular mode of administration. It will be appreciated that the unit content of active ingredients contained in an individual dose of each dosage form need not in itself constitute an effective amount, as the necessary effective amount could be reached by administration of a number of individual doses. The selection of dosage depends upon the dosage form utilized, the condition being treated, and the particular purpose to be achieved according to the determination of those skilled in the art.

The dosage regimen for treating a disease condition with the compounds and/or compositions of this invention is selected in accordance with a variety of factors, including the type, age, weight, sex, diet and medical condition of the patient, the route of administration, pharmacological considerations such as the activity, efficacy, pharmacokinetic and toxicology profiles of the particular compound employed, whether a drug delivery system is utilized and whether the compound is administered as part of a drug combination. Thus, the dosage regimen actually employed may vary widely and therefore may deviate from the preferred dosage regimen set forth above.

The following examples are offered in order to illustrate but not to limit the present invention.

### EXAMPLES

### Example 1

### Effect of SOD Mimic M40403 on IL-2 Induced Hypotension

IL-2, M40403 and IL-2/M40403 were administered to mice (n=6 per group) in order to determine the effect of these compounds on mean blood pressure. 180,000 IU of IL-2 was administered intraperitoneally (i.p.) twice daily for 5 days (10 doses) to each animal of the first group on a daily basis for 4 days. Experimental animals received M40403 (0.03-3 mg/kg) i.p.b.i.d. in conjunction with IL-2. Parallel groups of untreated mice or mice receiving M40403 alone (at the highest dose tested of 3 mg/kg) served as a control. Two hours after the last dose of IL-2, systolic blood pressure was measured via tail cuff (Stoelting, Wood Dell, Ill.) and using a PowerLab digital signal transducer (AD instruments, Mountain View, Calif.). Analysis was performed using Chart 3.6.1 software (AD instruments). The results of these tests can be found in Table 1.

TABLE 1
Protective effects of M40403 on IL-2 induced hypotension

| Group | Mean Systolic BP | Standard Deviation |
|---|---|---|
| Control | 82.6 | 3.47 |
| IL-2 | 44.53 | 7.85 |
| SOD mimic | 111.21 | 45.31 |
| IL-2 + SOD mimic | 114.39 | 29.4 |

The blood pressure values represent the mean arterial blood pressure from the mean values of the six mice of each group. This experiment demonstrated striking (approximately 50%) reduction of systolic BP following IL-2 administration (mean 45±8.0 mmHg). Mice receiving the SOD mimic alone had variable increases in BP compared to controls (mean 111±45 mmHg versus 83±4 mmHg-mostly error due to one very hypertensive mouse in SOD mimic group). Mice receiving the SOD mimic plus IL-2 had complete reversal of IL-2 induced hypotension (114±29 mmHg).

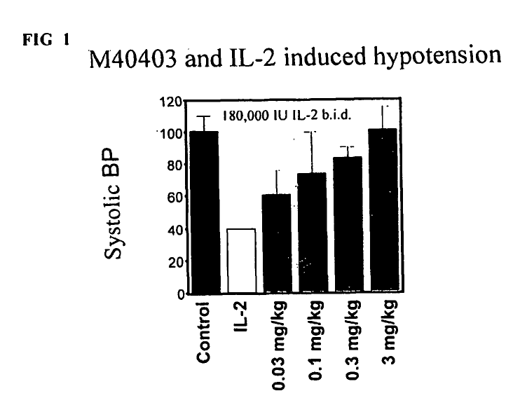

As demonstrated in FIG. 1, IL-2 induced hypotension was attenuated in a dose-dependent manner by M40403 with maximal reversal obtained at 3 mg/kg M40403. At the highest dose of M40403 (3 mg/kg, b.i.d. for 5 days) there were no apparent signs of toxicity and particularly no effect on systolic blood pressure.

### Example 2

### Dose Escalation Studies

The following studies were conducted to determine whether in addition to decreasing toxicity from IL-2, M40403 could be useful to increase the dose of IL-2 that can be tolerated by patients, or to assure that the full planned number of doses can be delivered, maximizing the potential for response.

Mice (n=6 per group) were treated with escalating doses of IL-2 of 180,000, 200,000, 250,000, 300,000, 350,000 and 400,000 IU IL-2 injected i.p. twice daily for 5 days (10 doses). Experimental animals received M40403 (3 mg/kg) i.p. b.i.d. in conjunction with IL-2. Two hours after the last dose of IL-2, systolic blood pressure was measured via tail cuff (Stoelting, Wood Dell, Ill.) and using a PowerLab digital signal transducer (AD instruments, Mountain View, Calif.). Analysis was performed using Chart 3.6.1 software (AD instruments).

In the absence of M40403, there was 1 death in each IL-2 treated group at 200,000, 250,000 and 300,000 I.U. b.i.d. (1 out 6 died), all mice died in the 400,000 I.U. b.i.d. group (6 deaths of 6 mice). In contrast, in the presence of M40403, all mice at all doses of IL-2 were alive (0 deaths of 6 mice in all groups). Accordingly, M40403 allowed BP to be maintained while IL-2 mediated death rates were reduced. Thus, higher doses of IL-2 may be safely administered in the presence of M40403.

### Example 3

### Evaluation of the Effect of M40403 on LAK Cell Induction In Vitro

Studies were conducted to determine if M40403 affected the ability of IL-2 to induce LAK cells cytotoxicity in vitro. Murine splenocytes were incubated with increasing concentrations of M40403 during IL-2 activation (6000 IU/ml rhIL-2 in RPMI-1640 medium containing 10% fetal calf serum, antibiotics, and 1 mM glutamine). Non-M40403 exposed cultures served as a positive control. After 72 h, LAK cells were harvested and cell viability evaluated. LAK cells cytotoxicity was tested against RD-995 tumor in triplicate samples at varying effector to target cell ratios (expressed as mean±SD) using previously published methods (Yim et al., 1994; Samlowski et al., 1998).

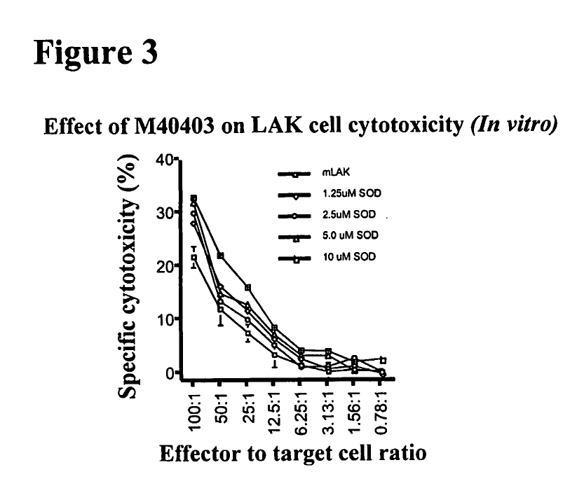

These experiments established that a broad range of M40403 concentrations (0-10 μM) did not adversely affect murine (mouse) LAK cell activation (See FIG. 3). In fact, a dose-dependent increase of LAK cell activation appeared to be induced. Cell viability was assessed by trypan blue exclusion. Viability remained at 100% of control at up to 10 μM M40403 (not shown). Similar results were obtained with M40403 when using human LAK cells (not shown).

### Example 4

### Evaluation of the Effect of M40403 on LAK Cell Induction In Vivo

Mice (n=6 per group) were treated with 180,000 IU IL-2 injected i.p. twice daily for 5 days (10 doses). Experimental animals received M40403 (3 mg/kg) i.p. b.i.d. in conjunction with IL-2. Parallel groups of untreated mice or mice receiving M40403 alone (at the highest dose tested of 3 mg/kg) served as a control. Mice were sacrificed 2 hours following the last dose of IL-2. A single cell suspension was prepared from the lymph nodes and spleen from each animal, and serial dilutions made (starting from 10⁶). Cytolytic activity of lymph node and spleen cells was tested against ⁵¹Cr-radiolabeled RD-995 tumor (NK resistant, LAK sensitive, 10⁴ cells) in a standard 4h ⁵¹Cr release assay at a varying effector to target cell ratio (in triplicate). Parallel wells, containing 6000 IU/ml IL-2 were set up, to test for rapid up-regulation of LAK activity which is found when IL-2 primed cytolytic effector cells are present. Statistical comparison of specific cytotoxicity (e.g. at 100:1 effector to target ratio) and lytic units per organ will be performed using Student's t-test, using Bonferroni's correction for multiple analyses.

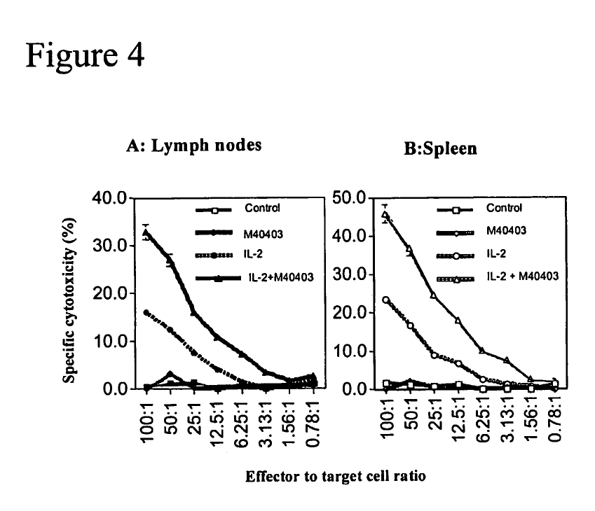

As can be seen in FIG. 4, M40403 significantly potentiated IL-2 induced LAK cells activation in vivo as seen in the lymph nodes (A) and spleen (B).

### Example 5

### Enhancement of IL-2 Induced Tumor Responses by M40403 In Vivo

The effects of M40403 on IL-2 induced tumor responses were assessed in two different tumors at an advanced stage of progression. RD-995 and Meth A have been chosen for initial experiments, because these tumors show differing sensitivity to IL-2 induced LAK cells, thus, allowing us to evaluate LAK mediated from non-LAK cell mediated tumor cell killing mechanisms induced by IL-2.

Rosenberg Tumor Model.

RD-995 is a skin fibrosarcoma that was induced by chronic ultraviolet light exposure of a C3H/HeN mouse (Samlowski et al., 1995). This tumor grows in non-immunosuppressed mice, and is thus termed a “progressor” tumor. RD-995 is sensitive to LAK cell mediated killing in vitro. Pulmonary metastases derived from RD-995 are exquisitely sensitive to IL-2 treatment (with or without infusion of LAK cells). This model (known as the Rosenberg tumor model) was used in the following experiment to establish that M40403 does not diminish IL-2 induced responses in mice.

Four groups (n=6 mice per group) of mice (control, M40403 alone, IL-2 only, IL-2+M40403) were acclimated to metabolic cages. Once stabilized, mice were injected with 10⁶ RD-995 tumor cells intravenously. An IL-2 only treatment group served as a positive control, with IL-2 treatment beginning 3 days after tumor injection (IL-2 given at 180,000 IU i.p. every 12 h for 5 days). Simultaneously, parallel groups of mice began M40403 (3 mg/kg), concurrently with IL-2. On day 14, mice were sacrificed by anesthetic overdose. After anterior thoracotomy, India ink solution (30 ml India ink, 4 drops ammonium hydroxide in 170 ml distilled water) was instilled into the trachea of each mouse via 18 ga. needle, until the lungs were completely expanded. Lungs were then excised and bleached in 5-10 ml Fekete's solution (100 ml 70% ethanol, 10 ml formaldehyde, 5 ml glacial acetic acid) for at least 24 h prior to enumeration of metastases using a magnifying lens.

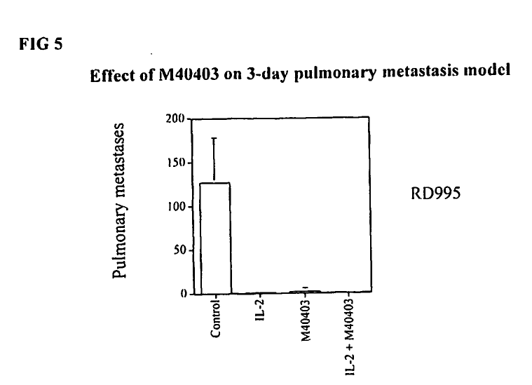

IL-2 treatment of mice eliminated the number of tumor nodules in lungs (FIG. 5). M40403 did not affect the anti-tumor effects of IL-2 (FIG. 5). However, M40403 by itself at the dose used completely eliminated the numbers of tumor nodules in lungs of mice (FIG. 5), an effect which was independent from LAK cell activation since it was found that M40403 by itself did not increase LAK cell induction in vivo (see FIG. 4). Based on the experimental data, Applicants believe that additive or synergistic effects of IL-2 plus M40403 are observed.

Meth A Fibrosarcoma Tumor Model

Meth A was induced in a BALB/c mouse by methylcholanthrene treatment (obtained from Lloyd Old, MSKCC). This very aggressive fibrosarcoma is one of the rare tumors resistant to LAK cell mediated cytolysis in vitro. When injected intraperitoneally into mice (malignant ascites), this tumor responds to IL-2 therapy with improved survival. The mechanism is not completely established but may involve nitric oxide as a component of the treatment response. See Yim et al., J. Immunol. 155: 4382-4390 (1995). M40403, does not inhibit NO and does not affect NO-mediated apoptosis of Meth A tumor (not shown). The Meth A model serves to provide a resistant tumor model to evaluate potentially synergistic antitumor effects of combinations of IL-2 with M40403. This model is more representative of the majority of IL-2 treated human cancers, which at best undergo disease stabilization following IL-2 treatment followed by eventual disease progression.

Groups of 8 normal BALB/c mice (Control, M40403 only, IL-2 only, IL-2+M40403) were injected with 2×10⁶ Meth A tumor cells intraperitoneally (day 0). On day 6 following tumor implantation, mice began treatment with IL-2 (180,000 IU s.c. every 12 h for 5 days) with or without concomitant treatment with M40403 (3 mg/kg, s.c.). Survival of the mice was used as the major endpoint to assess therapeutic responses.

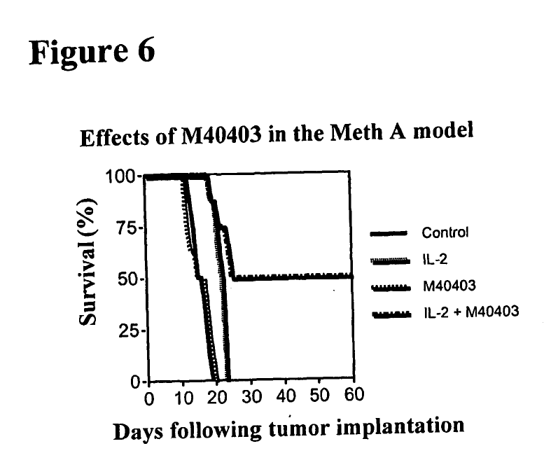

As shown in FIG. 6, a modest but definite survival advantage occurs in IL-2 treated mice as compared to controls (14 versus 19 days). M40403 itself did not affect survival. In contrast, there was marked synergy between IL-2 and M40403, with 50% of mice still alive on day 60 with no visible (apparent) ascites. This result indicates that M40403 not only blocks IL-2 induced hypotension but also improves IL-2 induced anti-tumor responses in a highly refractory treatment model thereby enhancing the therapeutic effects of IL-2.

### Example 5

### M40401 and IL-2

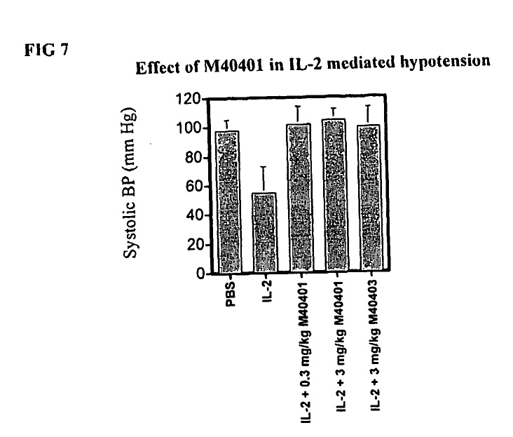

Preliminary experiments were also performed with M40401. These experiments demonstrated that M40401 does not interfere with the ability of IL-2 to activate LAK cells in vitro. Furthermore, and as expected from our previous data in septic shock, M40401 reversed IL-2 mediated hypotension at lower doses than M40403. Thus, full reversal of hypotension was seen at 0.3 mg/kg (b.i.d with IL-2 for 5 days, protocol identical to the one used for M40403) (See FIG. 7).

Other features, objects and advantages of the present invention will be apparent to those skilled in the art. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the present invention.

1. A method for inhibiting a fall in mean arterial pressure in a mammal due to the administration of a cytokine, the method comprising co-administering to the mammal a mean arterial pressure sustaining amount of a catalyst for the dismutation of superoxide in conjunction with a cytokine.

2. The method of claim 1 wherein inhibition of the fall in mean arterial pressure is achieved by limiting autooxidation of catecholamines.

3. The method of claim 1 wherein the catalyst is a non-proteinaceous catalyst, and the non-proteinaceous catalyst comprises an organic ligand chelated to a cation selected from the group of manganese(II), manganese(III), iron(II) and iron(III).

4. The method of claim 3, wherein the catalyst is a pentaaza-macrocyclic ligand complex.

5. The method of claim 4 wherein the pentaaza-macrocyclic ligand complex is selected from the group consisting of manganese and iron chelates of pentaazacyclopentadecane compounds, which are represented by the following formula: 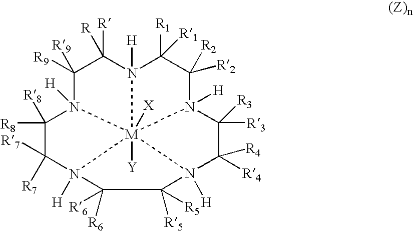
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈, and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ-M-(CH₂)_{w}-L-(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxarnic acid, thiotosylate, and anions of ion exchange resins.

6. The method of claim 3, wherein the catalyst is a porphyrin ligand complex or a substituted porphyrin ligand complex.

7. The method of claim 6 wherein the porphyrin ligand complex is selected from the group consisting of manganese (II) porphyrin complexes, manganese(III) porphyrin complexes, iron (II) porphyrin complexes, and iron(III) porphyrin complexes.

8. The method of claim 7 wherein the porphyrin ligand complex is a 5,10,15,20-tetrakis (2,4,6-trimethyl-3,5-disulfonatophenyl)-porphyrinato iron (III) (FeTMPS).

9. The method of claim 1, wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

10. The method of claim 9 wherein the cytokine is interleukin-2.

11. The method of claim 1, wherein the catalyst is administered before or after the administration of the cytokine.

12. The method of claim 1, wherein the catalyst is administered contemporaneously with the cytokine.

13. The method of any one of claims 1-12 wherein the method further comprises co-administering a catecholamine pressor agent.

14. The method of claim 13 wherein the catecholamine pressor agent is selected from the group consisting of dopamine, norepinephrine and epinephrine.

15. The method of claim 14 wherein the administration is carried out by intraarterial, intravenous, intramuscular or subcutaneous injection.

16. The method of claim 14 wherein the mammal is a human.

17. The method of claim 14 wherein the mammal is a companion pet.

18. The method of claim 14 wherein the mammal is a large non-human animal.

19. A method for increasing mean arterial pressure in a mammal suffering from hypotension resulting from administration of a cytokine, the method comprising co-administering to the mammal a mean arterial pressure increasing amount of a catalyst for the dismutation of superoxide in conjunction with a cytokine.

20. The method of claim 19 wherein the increase in mean arterial pressure is achieved by limiting autooxidation of catecholamines.

21. The method of claim 19 wherein the catalyst is a non-proteinaceous catalyst, and the non-proteinaceous catalyst comprises an organic ligand chelated to a cation selected from the group of manganese(II), manganese(III), iron(II) and iron(III).

22. The method of claim 21, wherein the catalyst is a pentaaza-macrocyclic ligand complex.

23. The method of claim 22 wherein the pentaaza-macrocyclic ligand complex is selected from the group consisting of manganese and iron chelates of pentaazacyclopentadecane compounds, which are represented by the following formula: 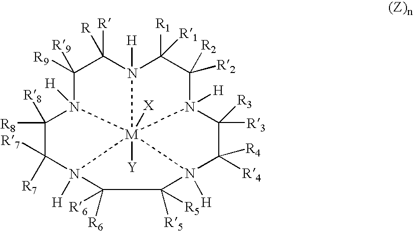
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₁, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

24. The method of claim 21, wherein the catalyst is a porphyrin ligand complex or a substituted porphyrin ligand complex.

25. The method of claim 24 wherein the porphyrin ligand complex is selected from the group consisting of manganese (II) porphyrin complexes, manganese(III) porphyrin complexes, iron (II) porphyrin complexes, and iron(III) porphyrin complexes.

26. The method of claim 25 wherein the porphyrin ligand complex is a 5,10,15,20-tetrakis(2,4,6-trimethyl-3,5-disulfonatophenyl)-porphyrinato iron (III) (FeTMPS).

27. The method of claim 19, wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

28. The method of claim 27 wherein the cytokine is interleukin-2.

29. The method of claim 19, wherein the catalyst is administered before or after administration of the cytokine.

30. The method of claim 27, wherein the catalyst is administered contemporaneously with the cytokine.

31. The method of any one of claims 19-30 wherein the method further comprises co-administering a catecholamine pressor agent.

32. The method of claim 31 wherein the catecholamine pressor agent is selected from the group consisting of dopamine, norepinephrine and epinephrine.

33. The method of claim 32 wherein the administration is carried out by intraarterial, intravenous, intramuscular or subcutaneous injection.

34. The method of claim 33 wherein the mammal is a human.

35. The method of claim 33 wherein the mammal is a companion pet.

36. The method of claim 33 wherein the mammal is a large veterinary animal.

37. A method for enhancing a mammal's immune response to cancer therapy, the method comprising administering to the mammal in conjunction with cytokine therapy, a therapeutically effective amount of a catalyst for the dismutation of superoxide.

38. The method of claim 37 wherein the catalyst is a non-proteinaceous catalyst, and the non-proteinaceous catalyst comprises an organic ligand chelated to a cation selected from the group of manganese(II), manganese(III), iron(II) and iron(III).

39. The method of claim 38, wherein the catalyst is a pentaaza-macrocyclic ligand complex.

40. The method of claim 39, wherein the pentaaza-macrocyclic ligand complex is selected from the group consisting of manganese and iron chelates of pentaazacyclopentadecane compounds, which are represented by the following formula: 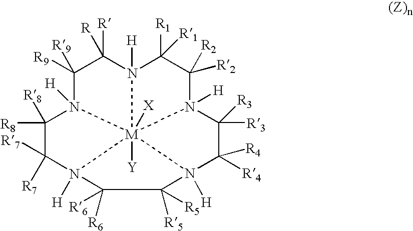
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉ and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈, and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

41. The method of claim 38, wherein the catalyst is a porphyrin ligand complex or a substituted porphyrin ligand complex.

42. The method of claim 41 wherein the porphyrin ligand complex is selected from the group consisting of manganese (II) porphyrin complexes, manganese(III) porphyrin complexes, iron (II) porphyrin complexes, and iron(III) porphyrin complexes.

43. The method of claim 42 wherein the porphyrin ligand complex is a 5,10,15,20-tetrakis(2,4,6-trimethyl-3,5-disulfonatophenyl)-porphyrinato iron (II) (FeTMPS).

44. The method of claim 37 wherein the method further comprises co-administering a cytokine selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

45. The method of claim 44 wherein the cytokine is interleukin-2.

46. The method of claim 44 wherein the catalyst is administered before or after the administration of the cytokine.

47. The method of claim 44 wherein the catalyst is administered contemporaneously with the cytokine.

48. The method of any one of claims 37-47 wherein the method further comprises co-administering a catecholamine pressor agent.

49. The method of claim 48 wherein the catecholamine pressor agent is selected from the group consisting of dopamine, norepinephrine and epinephrine.

50. The method of claim 49 wherein the administration is carried out by intraarterial, intravenous, intramuscular or subcutaneous injection.

51. The method of claim 49, wherein the mammal is a human.

52. The method of claim 49 wherein the mammal is a companion pet.

53. The method of claim 49 wherein the mammal is a large veterinary animal.

54. A composition comprising a cytokine, a catalyst for the dismutation of superoxide and a pharmaceutically acceptable carrier.

55. The composition of claim 54 wherein the catalyst is a non-proteinaceous catalyst, and the non-proteinaceous catalyst comprises an organic ligand chelated to a cation selected from the group of manganese(II), manganese(III), iron(II) and iron(III).

56. The composition of claim 55, wherein the catalyst is a pentaaza-macrocyclic ligand complex.

57. The composition of claim 56 wherein the pentaaza-macrocyclic ligand complex is selected from the group consisting of manganese and iron chelates of pentaazacyclopentadecane compounds, which are represented by the following formula: 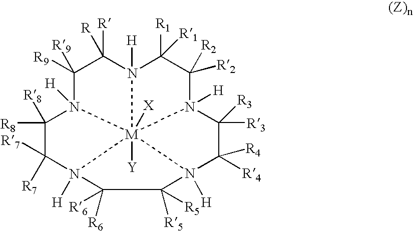
wherein M is a cation of a transition metal, preferably manganese or iron; wherein R, R′, R₁, R′₁, R₂, R′₂. R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

58. The composition of claim 55, wherein the catalyst is a porphyrin ligand complex or a substituted porphyrin ligand complex.

59. The composition of claim 58 wherein the porphyrin ligand complex is selected from the group consisting of manganese (II) porphyrin complexes, manganese(III) porphyrin complexes, iron (II) porphyrin complexes, and iron(III) porphyrin complexes.

60. The method of claim 59 wherein the porphyrin ligand complex is a 5,10,15,20-tetrakis(2,4,6-trimethyl-3,5-disulfonatophenyl)-porphyrinato iron (III) (FeTMPS).

61. The composition of claim 54 wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

62. The compositon of claim 61 wherein the cytokine is interleukin-2.

63. The composition of any one of claims 54-62 wherein the composition further comprises a catecholamine pressor agent.

64. The composition of claim 63 wherein the catecholamine pressor agent is selected from the group consisting of dopamine, norepinephrine and epinephrine.

65. A method of inhibiting the proliferation of a tumor in a mammal comprising administering a composition comprising a therapeutically effective amount of a catalyst for the dismutation of superoxide and a cytokine.

66. The method of claim 65 wherein the catalyst is a non-proteinaceous catalyst, and the non-proteinaceous catalyst comprises an organic ligand chelated to a cation selected from the group of manganese(II), manganese(III), iron(II) and iron(III).

67. The method of claim 66, wherein the catalyst is a pentaaza-macrocyclic ligand complex.

68. The method of claim 67 wherein the pentaaza-macrocyclic ligand complex is selected from the group consisting of manganese and iron chelates of pentaazacyclopentadecane compounds, which are represented by the following formula: 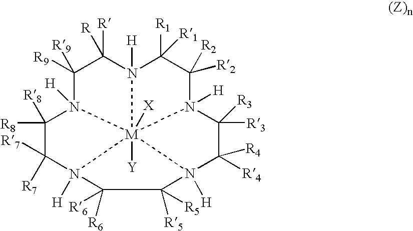
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆ R₇, R′₇, R₈, R′₈, R₉, and R′₉ which is attached to a different carbon atom in the macrocyclic ligand may be bound to form a strap represented by the formula:
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

69. The method of claim 68 wherein the catalyst is

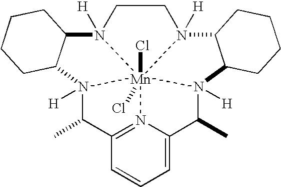

70. The method of claim 68 wherein the catalyst is

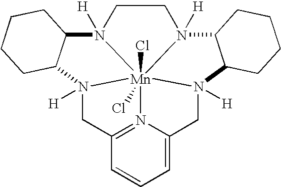

71. The method of claim 66, wherein the catalyst is a porphyrin ligand complex or a substituted porphyrin ligand complex.

72. The method of claim 71 wherein the porphyrin ligand complex is selected from the group consisting of manganese (II) porphyrin complexes, manganese(III) porphyrin complexes, iron (II) porphyrin complexes, and iron(III) porphyrin complexes.

73. The method of claim 72 wherein the porphyrin ligand complex is a 5,10,15,20tetrakis(2,4,6-trimethyl-3,5-disulfonatophenyl)-porphyrinato iron (III) (FeTMPS).

74. The method of claim 65 wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

75. The method of claim 74 wherein the cytokine is interleukin-2.

76. The method of claim 74 wherein the catalyst is administered before or after the administration of the cytokine.

77. The method of claim 74 wherein the catalyst is administered contemporaneously with the cytokine.

78. A method of enhancing a cytokine-mediated immune response against a virus in a mammal comprising administering a composition comprising a therapeutically effective amount of a catalyst for the dismutation of superoxide and a cytokine

79. The method of claim 78 wherein said composition further comprises a cytokine selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

80. The method of claim 79 wherein said virus is a hepatitis C virus.

81. The method of claim 80 wherein the cytokine is interferon-alpha (IF-α).

82. The method of claim 79 wherein said virus is a human immunodeficiency virus.

83. The method of claim 82 wherein the cytokine is interleukin-2 (IL-2).

84. The method of any one of claims 78-83 wherein the catalyst is a non-proteinaceous catalyst, and the non-proteinaceous catalyst comprises an organic ligand chelated to a cation selected from the group of manganese(II), manganese(III), iron(II) and iron(III).

85. The method of claim 84 wherein the catalyst is a pentaaza-macrocyclic ligand complex.

86. The method of claim 85 wherein the pentaaza-macrocyclic ligand complex is selected from the group consisting of manganese and iron chelates of pentaazacyclopentadecane compounds, which are represented by the following formula: 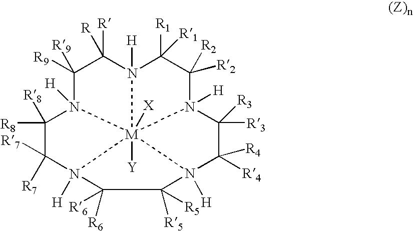
wherein M is a cation of a transition metal, preferably manganese or iron; wherein R, R′, R₁, R′₁, R₂, R′₂, R₃, R′₃, R′₄, R₅, R′₅, R₆, R′₆, R₇, R′₇, R₈, R′₈, R₉ and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅, and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉ and R′₉ together with a different one of R, R′, R₁, R′₁, R₂, R′₂, R₃, R′₃, R₄, R′₄, R₅, R′₅, R₆, R′₆, R₇, R′₇, R₈, R′₈, R₉, and R′₉ which is attached to a different carbon atom in the macrocyclic ligand may be bound to form a strap represented by the formula:
—(CH₂)ₓ-M—(CH₂)_{w}-L—(CH₂)₂—I—(CH)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

87. The method of claim 86 wherein the catalyst is

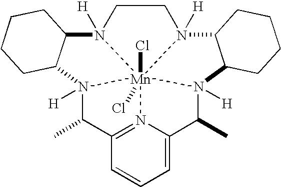

88. The method of claim 86 wherein the catalyst is

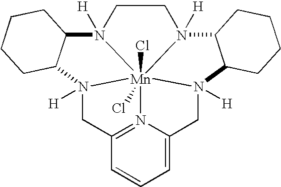

89. The method of claim 85, wherein the catalyst is a porphyrin ligand complex or a substituted porphyrin ligand complex.

90. The method of claim 89 wherein the porphyrin ligand complex is selected from the group consisting of manganese (II) porphyrin complexes, manganese(III) porphyrin complexes, iron (II) porphyrin complexes, and iron(II) porphyrin complexes.

91. The method of claim 90 wherein the porphyrin ligand complex is a 5,10,15,20-tetrakis(2,4,6-trimethyl-3,5-disulfonatophenyl)-porphyrinato iron (III) (FeTMPS).

92. A method of treating hepatitis C in a mammal comprising administering to the mammal a composition comprising a catalyst for the dismutation of superoxide and a cytokine.

93. The method of claim 92 wherein the catalyst is a non-proteinaceous catalyst, and the non-proteinaceous catalyst comprises an organic ligand chelated to a cation selected from the group of manganese(II), manganese(III), iron(II) and iron(III).

94. The method of claim 93 wherein the catalyst is a pentaaza-macrocyclic ligand complex.

95. The method of claim 94 wherein the pentaaza-macrocyclic ligand complex is selected from the group consisting of manganese and iron chelates of pentaazacyclopentadecane compounds, which are represented by the following formula: 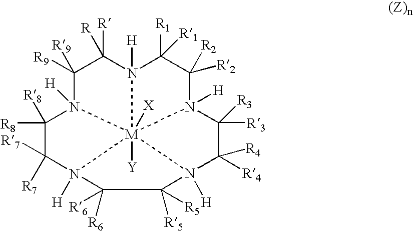
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉, and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁, or R′₁, and R₂ or R′₂, R₃ or R′₃ and R₄ or R′₄, R₅ or R′₅ and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R₉, and R′₉ together with a different one of R, R′, R₁, R′₁, R₂, R′₂, R₃, R′₃, R₄, R′₄, R₅, R′₅, R₆, R′₆. R₇, R′₇, R₈, R′₈, R₉, and R′₉ which is attached to a different carbon atom in the macrocyclic ligand may be bound to form a strap represented by the formula:
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphoniurn, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

96. The method of claim 95 wherein the catalyst is

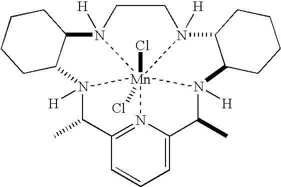

97. The method of claim 95 wherein the catalyst is

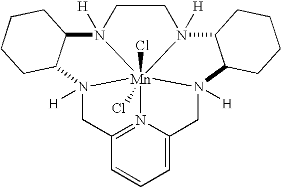

98. The method of claim 94, wherein the catalyst is a porphyrin ligand complex or a substituted porphyrin ligand complex.

99. The method of claim 98 wherein the porphyrin ligand complex is selected from the group consisting of manganese (II) porphyrin complexes, manganese(III) porphyrin complexes, iron (II) porphyrin complexes, and iron(III) porphyrin complexes.

100. The method of claim 99 wherein the porphyrin ligand complex is a 5,10,15,20-tetrakis(2,4,6-trimethyl-3,5-disulfonatophenyl)-porphyrinato iron (III) (FeTMPS).

101. The method of claim 94, wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

102. The method of claim 101 wherein the cytokine is interferon-alpha.

103. A method of treating a mammal infected with HIV or suffering from AIDS comprising administering to the mammal a composition comprising a catalyst for the dismutation of superoxide and a cytokine.

104. The method of claim 103 wherein the catalyst is a non-proteinaceous catalyst, and the non-proteinaceous catalyst comprises an organic ligand chelated to a cation selected from the group of manganese(II), manganese(III), iron(II) and iron(III).

105. The method of claim 104 wherein the catalyst is a pentaaza-macrocyclic ligand complex.

106. The method of claim 105 wherein the pentaaza-macrocyclic ligand complex is selected from the group consisting of manganese and iron chelates of pentaazacyclopentadecane compounds, which are represented by the following formula: 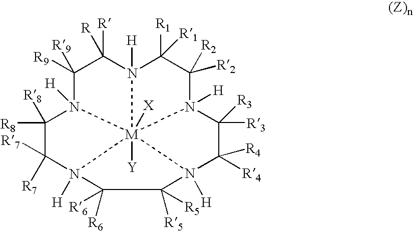
wherein M is a cation of a transition metal, preferably manganese or iron; wherein R, R′, R₁, R′₁, R₂, R′₂, R₃, R′₃, R₄, R′₄, R₅, R′₅, R₆, R′₆, R₇, R′₇, R₈, R′₈, R₉ and R′₉ independently represent hydrogen, or substituted or unsubstituted alkyl, alkenyl, alkynyl, cycloalkyl, cycloalkenyl, cycloalkylalkyl, cycloalkylcycloalkyl, cycloalkenylalkyl, alkylcycloalkyl, alkylcycloalkenyl, alkenylcycloalkyl, alkenylcycloalkenyl, heterocyclic, aryl and aralkyl radicals; R₁ or R′₁ and R₂ or R′₂, R₃ or R′₃ and R₄ or R′₄, R₅ or R′₅, and R₆ or R′₆, R₇ or R′₇ and R₈ or R′₈, and R₉ or R′₉ and R or R′ together with the carbon atoms to which they are attached independently form a substituted or unsubstituted, saturated, partially saturated or unsaturated cyclic or heterocyclic having 3 to 20 carbon atoms; R or R′ and R₁ or R′₁, R₂ or R′₂ and R₃ or R′₃, R₄ or R′₄ and R₅ or R′₅, R₆ or R′₆ and R₇ or R′₇, and R₈ or R′₈ and R₉ or R′₉ together with the carbon atoms to which they are attached independently form a substituted or unsubstituted nitrogen containing heterocycle having 2 to 20 carbon atoms, provided that when the nitrogen containing heterocycle is an aromatic heterocycle which does not contain a hydrogen attached to the nitrogen, the hydrogen attached to the nitrogen as shown in the above formula, which nitrogen is also in the macrocyclic ligand or complex, and the R groups attached to the included carbon atoms of the macrocycle are absent; R and R′, R₁ and R′₁, R₂ and R′₂, R₃ and R′₃, R₄ and R′₄, R₅ and R′₅, R₆ and R′₆, R₇ and R′₇, R₈ and R′₈, and R₉ and R′₉, together with the carbon atom to which they are attached independently form a saturated, partially saturated, or unsaturated cyclic or heterocyclic having 3 to 20 carbon atoms; and one of R, R′, R₁, R′₁, R₂, R′₂, R₃, R′₃, R₄, R′₄, R₅, R′₅, R₆, R′₆, R₇, R′₇, R₈, R′₈, R′₉, and R′₉ together with a different one of R, R′, R₁, R′₁, R₂, R′₂, R₃, R′₃, R₄, R′₄, R₅, R′₅, R₆, R′₆. R₇, R′₇, R₈, R′₈, R₉, and R′₉ which is attached to a different carbon atom in the macrocyclic ligand may be bound to form a strap represented by the formula:
—(CH₂)ₓ-M—(CH₂)_{w}-L—(CH₂)_{z}—I—(CH₂)_{y}—
wherein w, x, y and z independently are integers from 0 to 10 and M, L and I are independently selected from the group consisting of alkyl, alkenyl, alkynyl, aryl, cycloalkyl, heteroaryl, alkaryl, alkheteroaryl, aza, amide, ammonium, oxa, thia, sulfonyl, sulfinyl, sulfonamide, phosphoryl, phosphinyl, phosphino, phosphonium, keto, ester, alcohol, carbamate, urea, thiocarbonyl, borates, boranes, boraza, silyl, siloxy, silaza and combinations thereof; and combinations thereof;
and wherein X, Y and Z are independently selected from the group consisting of halide, oxo, aquo, hydroxo, alcohol, phenol, dioxygen, peroxo, hydroperoxo, alkylperoxo, arylperoxo, ammonia, alkylamino, arylamino, heterocycloalkyl amino, heterocycloaryl amino, amine oxides, hydrazine, alkyl hydrazine, aryl hydrazine, nitric oxide, cyanide, cyanate, thiocyanate, isocyanate, isothiocyanate, alkyl nitrile, aryl nitrile, alkyl isonitrile, aryl isonitrile, nitrate, nitrite, azido, alkyl sulfonic acid, aryl sulfonic acid, alkyl sulfoxide, aryl sulfoxide, alkyl aryl sulfoxide, alkyl sulfenic acid, aryl sulfenic acid, alkyl sulfinic acid, aryl sulfinic acid, alkyl thiol carboxylic acid, aryl thiol carboxylic acid, alkyl thiol thiocarboxylic acid, aryl thiol thiocarboxylic acid, alkyl carboxylic acid (such as acetic acid, trifluoroacetic acid, oxalic acid), aryl carboxylic acid (such as benzoic acid, phthalic acid), urea, alkyl urea, aryl urea, alkyl aryl urea, thiourea, alkyl thiourea, aryl thiourea, alkyl aryl thiourea, sulfate, sulfite, bisulfate, bisulfite, thiosulfate, thiosulfite, hydrosulfite, alkyl phosphine, aryl phosphine, alkyl phosphine oxide, aryl phosphine oxide, alkyl aryl phosphine oxide, alkyl phosphine sulfide, aryl phosphine sulfide, alkyl aryl phosphine sulfide, alkyl phosphonic acid, aryl phosphonic acid, alkyl phosphinic acid, aryl phosphinic acid, alkyl phosphinous acid, aryl phosphinous acid, phosphate, thiophosphate, phosphite, pyrophosphite, triphosphate, hydrogen phosphate, dihydrogen phosphate, alkyl guanidino, aryl guanidino, alkyl aryl guanidino, alkyl carbamate, aryl carbamate, alkyl aryl carbamate, alkyl thiocarbamate aryl thiocarbamate, alkyl aryl thiocarbamate, alkyl dithiocarbamate, aryl dithiocarbamate, alkyl aryl dithiocarbamate, bicarbonate, carbonate, perchlorate, chlorate, chlorite, hypochlorite, perbromate, bromate, bromite, hypobromite, tetrahalomanganate, tetrafluoroborate, hexafluorophosphate, hexafluoroantimonate, hypophosphite, iodate, periodate, metaborate, tetraaryl borate, tetra alkyl borate, tartrate, salicylate, succinate, citrate, ascorbate, saccharinate, amino acid, hydroxamic acid, thiotosylate, and anions of ion exchange resins.

107. The method of claim 106 wherein the catalyst is

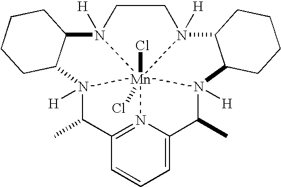

108. The method of claim 106 wherein the catalyst is

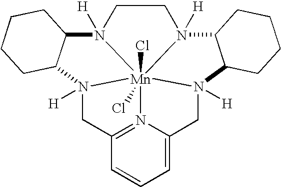

109. The method of claim 105 wherein the catalyst is a porphyrin ligand complex or a substituted porphyrin ligand complex.

110. The method of claim 109 wherein the porphyrin ligand complex is selected from the group consisting of manganese (II) porphyrin complexes, manganese(III) porphyrin complexes, iron (II) porphyrin complexes, and iron(III) porphyrin complexes.

111. The method of claim 110 wherein the porphyrin ligand complex is a 5,10,15,20-tetrakis(2,4,6-trimethyl-3,5-disulfonatophenyl)-porphyrinato iron (III) (FeTMPS).

112. The method of claim 105, wherein the cytokine is selected from the group consisting of interleukin-1, interleukin-2, interleukin-3, interleukin-4, interleukin-5, interleukin-6, interleukin-7, interleukin-10, interleukin-12, interleukin-15, gamma-interferon, alpha-interferon, tumor necrosis factor, granulocyte colony stimulating factor and granulocyte-macrophage colony stimulating factor.

113. The method of claim 112 wherein the cytokine is interferon-alpha.
